(19) United States
(12) Patent Application Publication
(10) Pub. No.: US 2003/0199003 A1
(43) Pub. Date: Oct. 23, 2003

(54) High-level production of amyloid-beta peptides from IMR-32 cells

(76) Inventors: Nancy C. Stratman, Kalamazoo, MI (US); Donald B. Carter, Kalamazoo, MI (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/369,940

(22) Filed: Feb. 20, 2003

Related U.S. Application Data
(60) Provisional application No. 60/359,943, filed on Feb. 27, 2002.

(51) Int. Cl. G01N 33/53; G01N 33/567; C12P 21/02; C12N 5/08; C07K 14/47

(52) U.S. Cl. 435/7.2; 435/69.1; 435/368; 435/320.1; 530/350

(57) ABSTRACT

Methods for producing peptides resulting from the proteolytic processing of amyloid precursor protein (APP). The methods involve high-density plating of IMR-32 cells. IMR-32 cells produced by the methods of the present invention are useful for screening for modulators of peptides resulting from proteolytic processing of APP, e.g., Aβ and sAPPα.

### RELATED APPLICATION DATA

This application claims priority to provisional application U.S. Ser. No. 60/359,943, filed on Feb. 27, 2002, the disclosure of which is incorporated by reference in its entirety.

### BACKGROUND

Alzheimer's disease (AD) causes progressive dementia with consequent formation of amyloid plaques, neurofibrillary tangles, gliosis and neuronal loss. A primary component of amyloid plaques is 40-42 amino acid peptides, referred to as amyloid beta (Aβ) peptide, resulting from the proteolytic processing of amyloid precursor protein (APP). APP is localized to the cell surface, has a single C-terminal transmembrane domain, and ranges in size from 695-770 amino acids in length. Processing of APP by α-secretase (FIG. 1, α site) releases a soluble, extracellular domain of APP from the cell surface, a process that is apparently nonpathogenic. This α secretase processing, creating soluble APP-α is normal and not thought to contribute to AD.

In contrast, sequential processing of APP at the β- and γ-secretase sites (FIG. 1, β and γ) releases the Aβ peptide. The Aβ peptide is derived from a region of APP adjacent to and containing a portion of the transmembrane domain. There are two predominant forms of Aβ peptide, Aβ₁₋₄₀ and Aβ₁₋₄₂. Typically, Aβ peptide-producing cells secrete both forms of the peptide with Aβ₁₋₄₂ comprising about 10% of the total Aβ peptide produced. Although it is the minor population, experimental evidence suggests that Aβ₁₋₄₂ is responsible for the formation of the amyloid plaques.

Enzymes that cleave at the α- and β-secretase sites are disclosed in International Patent Publication No. WO 01/23533 (Pharmacia & Upjohn Co.). This patent publication also describes methods of screening for compounds that modulate the activity of the enzymes. These and other in vitro or in vivo screening methods are useful in finding compounds that may be effective in treating AD.

Initially, in vitro models producing Aβ peptide consisted of cell lines transformed to recombinantly overexpress APP. However, because the cell lines did not have neuron- or glia-like properties, proteolytic processing in these transformed cell lines was aberrant. Subsequently, researchers have turned to wild-type cell lines with neuron- or glia-like properties to study APP processing. One such cell line is the wild-type human neuroblastoma cell line IMR-32 (Neill et al., J. Neurosci Res 39(4):482-93, 1994). IMR-32 cells have been shown to secrete Aβ₁₋₄₀ and Aβ₁₋₄₂. However, because the level of Aβ peptide secreted by IMR-32 cells is low, particularly Aβ₁₋₄₂, the cells must be cultured for extended periods (48 hours or greater) to allow sufficient accumulation within the medium to be detected using standard techniques. When using IMR-32 cells to study Aβ peptide production or screen compounds that affect its production, this extended culture requirement significantly decreases the rate of data production and analysis. Thus, a need exists for materials and methods that permit analysis of APP processing that avoid one or more of these shortcomings, to facilitate development of Alzheimer's Disease therapeutics.

### SUMMARY OF THE INVENTION

Here, described are materials and methods that significantly decrease the time required for detectable levels of Aβ peptide to accumulate in the media of cells used for analysis of APP processing, such as IMR-32 cells. This decrease in time significantly increases the rate in which data is produced in methodologies using IMR-32 cells for Aβ peptide production. This is particularly useful in high throughput screening methodologies. In addition, because compound stability may be compromised with longer incubation times, the decrease in time minimizes the presence of Aβ peptide decomposition products.

One aspect of the present invention is a method of culturing IMR-32 cells to increase production of peptides resulting from the proteolytic processing of amyloid precursor protein. This method includes steps of seeding a culture vessel with IMR-32 neuroblastoma cells at a density of at least about 5.0×10⁴ cells/cm² of growth area to about 1.0×10⁶ cells/cm² of growth area, growing the IMR-32 neuroblastoma cells in medium containing serum until confluent or near confluent, changing the medium to serum-free medium, and incubating the IMR-32 neuroblastoma in the serum-free medium.

In one embodiment, the IMR-32 neuroblastoma cells are seeded at a density of about 2.5×10⁵ to about 5×10⁵ cells/ml.

The amount of time the IMR-32 neuroblastoma cells are incubated in the serum-free medium may vary. In some embodiments, the incubation time is less than 24 hours. In other embodiments, the incubation time is between 4 and 24 hours. In yet other embodiments, the incubation time is less than 4 hours. In some embodiments, the incubation time is sufficient to create a high yield of amyloid beta peptides in the medium, such as at least 3000 pg/ml. Amyloid beta peptides can be obtained by collecting the serum free medium after the incubation. A measurement of the amount of amyloid beta peptides in the collected serum-free medium can be taken. In one embodiment, the serum-free medium is B27 supplemented medium.

Another aspect of the invention is a method of screening for a modulator of proteolytic processing of amyloid precursor protein. This method comprises the steps of seeding a culture vessel with NMR-32 neuroblastoma cells at a density of at least about 5.0×10⁴ cells/cm² of growth area to about 1.0×10⁶ cells/cm² of growth area, growing the IMR-32 neuroblastoma cells in medium containing serum, changing the medium to serum-free medium that comprises a candidate modulator of amyloid beta peptide, incubating the IMR-32 neuroblastoma cells in the serum-free medium, measuring at least one peptide produced by proteolytic processing of amyloid precursor protein present in the serum-free medium containing the candidate modulator, and comparing the measurement to a control measurement. This method may be used to find an inhibitor of the production of a particular APP-derived peptide. In certain embodiments, the concentration of one or more APP-derived peptides, such as Aβ₁₋₄₀, Aβ₁₋₄₂, or sAPPα, in the medium is determined. The control measurement may be a measurement obtained by incubating IMR-32 neuroblastoma cells in the absence of the candidate modulator.

Another aspect of the invention is a method of detecting an inhibitor of amyloid beta peptide production. This method comprises the steps of seeding a culture vessel with IMR-32 neuroblastoma cells at a density of at least about 5.0×10⁴ cells/cm² of growth area to about 1.0×10⁶ cells/cm² of growth area, growing the IMR-32 neuroblastoma cells in medium containing serum, changing the medium to serum-free medium comprising a first concentration of a candidate inhibitor of amyloid beta peptide production, incubating the IMR-32 neuroblastoma cells in the serum-free medium, measuring a first level of amyloid beta peptide in the serum-free medium containing the concentration of the candidate inhibitor, repeating, subsequently or in parallel; the previous steps one or more times wherein the concentration of the candidate inhibitor is different than that of the first concentration and a level of amyloid beta peptide is measured for each concentration, and comparing the level of amyloid beta peptide at two or more concentrations of the candidate inhibitor, wherein a lower level of amyloid beta peptide at a higher concentration of the candidate inhibitor indicates an inhibitor of amyloid beta peptide production. An inhibitor determined by this method may be formulated into a composition comprising an effective amount of the inhibitor and a pharmaceutical carrier. Furthermore, this composition may be administered to an animal and the animal evaluated to determine amyloid beta peptide production.

Information regarding whether a compound or composition inhibits amyloid beta peptide is useful and valuable to those in the field of producing inhibitors of amyloid beta peptide production and those wishing to treat disorders associated with amyloid beta peptide production, such as AD. Thus, in one aspect of the invention the results of one or more assays of the present invention are included in a report, such as a computer printout, electronic mail, oral presentation or recording, or visual presentation or recording.

In another embodiment, a cell line is produced using a method of the present invention. In one embodiment, the cell line so produced secretes amyloid beta peptides into the medium at a rate of at least 1300 pg/ml/day.

Additional features and variations of the invention will be apparent to those skilled in the art from the entirety of this application, and all such features are intended as aspects of the invention.

Likewise, features of the invention described herein can be re-combined into additional embodiments that also are intended as aspects of the invention, irrespective of whether the combination of features is specifically mentioned above as an aspect or embodiment of the invention. Also, only such limitations that are described herein as critical to the invention should be viewed as such; variations of the invention lacking limitations that have not been descried herein as critical are intended as aspects of the invention.

In addition to the foregoing, the invention includes, as an additional aspect, all embodiments of the invention narrower in scope in any way than the variations specifically mentioned above. Although the applicant(s) invented the full scope of the claims appended hereto, the claims appended hereto are not intended to encompass within their scope the prior art work of others. Therefore, in the event that statutory prior art within the scope of a claims is brought to the attention of the applicant(s) by a Patent Office or other entity or individual, the applicant(s) reserve the right to exercise amendment rights under applicable patent laws to redefine the subject matter of such claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWING

FIG. 1 depicts APP and the peptides that result from proteolytic processing of APP. Arrows point to the cleavage site of the α-, β-, and γ- secretases.

### DETAILED DESCRIPTION OF THE PREFERRED EMBODIMENT

The IMR-32 cell line (ATCC Number CCL-127) was derived from human neuroblastoma cells (Neill et al., J. Neurosci Res 39(4):482-93, 1994). IMR-32 cells have been shown to express APP and to secrete Aβ₁₋₄₀ and Aβ₁₋₄₂. Although cells that express Aβ₁₋₄₀ and Aβ₁₋₄₂ generally are useful in studies related to Alzheimer's disease, poor growth and low peptide secretion rates characteristic of IMR-32 cells have limited their use in Alzheimer's research.

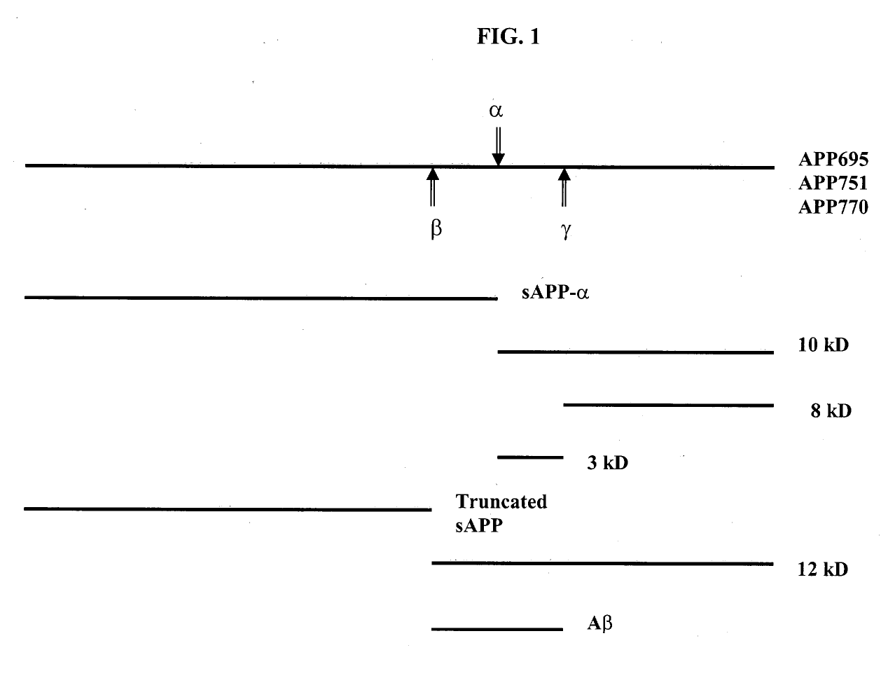

Described herein are methods of culturing IMR-32 cells. IMR-32 cells grown by the methods described herein are useful for analyzing APP processing and for producing peptides resulting from such proteolytic processing, including Aβ₁₋₄₀, Aβ₁₋₄₂, and soluble APP-α (sAPP-α). A schematic of APP processing is provided in FIG. 1.

Furthermore, IMR-32 cells grown by a method of the present invention are useful for screening for modulators of proteolytic processing of APP. Modulators found or characterized by the screening methods may be placed within a composition for use in methods of modulating APP processing, including treatment of amyloid disease.

Typically in the art, IMR-32 cells are plated at a low density, e.g., 2.3×10⁴ cells/cm², and allowed to grow into a monolayer before splitting the cells 1:3 to 1:6 and transferring to a fresh plate. Often cell lines do not react favorably to plating at higher densities, resulting in abundant cell death. Thus, such high-density seeding often is counter-productive.

The methods of the present invention include high-density seeding (plating) of IMR-32 cells. As described herein, high-density plating of IMR-32 cells does not lead to abundant cell death. Rather, surprisingly, high-density plating leads to increased yield of peptides resulting from the processing of APP.

To seed IMR-32 cells at a high density, the cells are added to a vessel (e.g., culture disk, flask, or well of a plate) at from about 5.0×10⁴ cells/cm² to about 1.0×10⁶ cells/cm², where cm² refers to the growth area of the vessel. For example, the growth area in a well of a conventional 96-well plate is about 0.3 cm², 24-well plate is about 1.9 cm², 12-well plate is about 3.7 cm², and 6-well plate is about 9.0 cm². The cells that are added to the vessel can be obtained from either a confluent or non-confluent culture of IMR-32 cells.

By “about,” it is meant that the number of cells is within the standard of error commonly accepted by those of skill in the art for a counting method. For example, if the accepted standard of error for a particular cell counting method is 10%, then “about 1.0×10⁶ cells/cm²ⁿ would include from 9.0×10⁵ cells/cm² to 1.1×10⁶ cells/cm².

In certain embodiments, the cells are added to the vessel at about 9.4×10⁴ cells/cm² to about 3.1×10⁵ cells/cm². In other embodiments, the cells are added to the vessel at about 9.4×10⁴ cells/cm² to about 1.6×10⁵ cells/cm². Of course, the addition of essentially any number of cells between 5.0×10⁴ cells/cm² to about 1.0×10⁶ cells/cm² will be considered high density seeding, including but not limited to about 5.1×10⁴ cells/cm², about 5.2×10⁴ cells/cm², about 5.3×10⁴ cells/cm², about 5.4×10⁴ cells/cm², about 5.5×10⁴ cells/cm², 5.6×10⁴ cells/cm², about 5.7×10⁴ cells/cm², about 5.8×10⁴ cells/cm², about 5.9×10⁴ cells/cm², about 6.0×10⁴ cells/cm², about 6.1×10⁴ cells/cm², about 6.2×10⁴ cells/cm², about 6.3×10⁴ cells/cm², about 6.4×10⁴ cells/cm², about 6.5×10⁴ cells/cm², 6.6×10⁴ cells/cm², about 6.7×10⁴ cells/cm², about 6.8×10⁴ cells/cm², about 6.9×10⁴ cells/cm², about 7.0×10⁴ cells/cm², about 7.1×10⁴ cells/cm², about 7.2×10⁴ cells/cm², about 7.3×10⁴ cells/cm², about 7.4×10⁴ cells/cm², about 7.5×10⁴ cells/cm², 7.6×10⁴ cells/cm², about 7.7×10⁴cells/cm², about 7.8×10⁴ cells/cm², about 7.9×10⁴cells/cm², about 8.0×10⁴ cells/cm², about 8.1×10⁴ cells/cm², about 8.2×10⁴ cells/cm², about 8.3×10⁴ cells/cm², about 8.4×10⁴ cells/cm², about 8.5×10⁴ cells/cm², 8.6×10⁴ cells/cm², about 8.7×10⁴ cells/cm², about 8.8×10⁴cells/cm², about 8.9×10⁴ cells/cm², about 9.0×10⁴ cells/cm², about 9.1×10⁴ cells/cm², about 9.2×10⁴ cells/cm², about 9.3×10⁴ cells/cm², about 9.4×10⁴ cells/cm², about 9.5×10⁴cells/cm², about 9.6×10⁴ cells/cm², about 9.7×10⁴ cells/cm², about 9.8×10⁴ cells/cm², about 9.9×10⁴ cells/cm², about 1.0×10⁵ cells/cm², about 1.1×10⁵ cells/cm², about 1.2×10⁵ cells/cm², about 1.3×10⁵ cells/cm², about 1.4×10⁵ cells/cm², about 1.5×10⁵ cells/cm², about 2.0×10⁵ cells/cm², about 3.0×10⁵ cells/cm², about 4.0×10⁵ cells/cm², about 5.0×10⁵ cells/cm², about 6.0×10⁵ cells/cm², about 7.0×10⁵ cells/cm², about 8.0×10⁵ cells/cm², about 9.0×10⁵ cells/cm², and about 1.0×10⁶ cells/cm².

Although vessels that may be easily manipulated are preferred in embodiments wherein the IMR-32 cells are used in screening assays, the invention is not limited by the size of the vessel. Larger vessels are useful when a large quantity of peptides is desired, e.g., large-scale purified peptide production. Methods of growing large quantities of mammalian cells for protein production are known in the art. (For a review, see Chu and Robinson, Current Opinion in Biotechnology 12:2:180-187, 2001.). Smaller vessels (e.g., the wells of a multiwell plate) are usually preferred for high throughput screening assays.

After high-density seeding, the IMR-32 cells are grown to confluency or near confluency in medium containing growth factors. Many different growth media are known in the art. An exemplary medium comprises minimum essential medium with Earle's salts, 10% fetal bovine serum, 2 mM L-glutamine, 100 U/ml penicillin, 100 μg/ml sptreptomycin, 1 mM sodium pyruvate, and 0.1 mM non-essential amino acids. However, the present invention is not limited by the type of media used to grow the IMR-32 cells, as long as the medium allows the high-density seeded IMR-32 cells to grow to confluency or near confluency. By near confluency, it is meant that the IMR-32 cells occupy at least 80 percent of the growth area of the vessel. The IMR-32 cells may occupy 85%, 90%, 95%, or 99% of the growth area when near confluent. Preferably, the IMR-32 cells are grown to confluency, occupying 100% of the growth area.

Examples of media that may be used to grow the IMR-32 cells to confluency or near confluency are MEM, DMEM, GMEM, RPMI 1640, and Ham F10 nutrient mixture. Additional components may be added to the medium. These components are well known in the art. Examples include antibiotics, amino acids, buffers, and growth factors.

Antibiotics are typically added to the medium to prevent bacterial contamination of the cultured cells. Although many different antibiotics may be used, some antibiotics affect the growth of the tissue culture cells themselves. Thus, it is optimal to choose antibiotics and/or antibiotic concentrations that have a minimal affect on the cultured cells. Examples of antibiotics that may be used include penicillin, streptomycin, amphotericin B, ampicillin, chloramphenicol, gentamicin, kanamycin, and tetracycline. For example, penicillin and streptomycin may be included in the medium at a concentration of 100 U/ml and 100 μg/ml, respectively.

Amino acids are often included in growth media. Cells cannot always produce all of the amino acids essential to their growth. Therefore, one or more essential amino acids are added to the growth medium. An example of an amino acid that is often added to growth media is L-glutamine. Also, non-essential amino acids may be included in the growth medium. Tissue culture grade essential and non-essential amino acids are commercially available.

Buffers are added to growth media to maintain pH. Buffers commonly used in tissue culture include bicarbonate, HEPES, PIPES, MOPS, and TES.

To proliferate in culture, tissue culture cells typically require the presence of certain proliferative agents such as cytokines or hormones in the medium. The appropriate proliferative agent(s) may be added individually to a medium. Alternatively, the proliferative agent(s) may be provided in the form of serum. An example of a commonly used serum is fetal bovine serum. To grow IMR-32 cells, the growth medium contains anywhere from about 5% to about 30% fetal bovine serum with 10% being preferred. Serum from many other sources may be used including human or horse. The serum may be heat inactivated prior to addition to the growth medium. One method of heat inactivating serum is to incubate the serum at 56° C. for 30 min.

Variations can also be made in the growth conditions and still remain within the scope of the invention. Examples of growth conditions that may be altered include temperature, CO₂ percentage, and O₂ percentage. In preferred embodiments, the IMR-32 cells are grown at 37° C. in 5% CO₂ with the balance being atmospheric air. In certain situations, it may be beneficial to grow the cells at physiologic or hypoxic oxygen concentrations. Such conditions and the methods of growing cells under such conditions are well known in the art. For example, WO 00/29549 discloses methods of growing neuronal cells under varying oxygen conditions including hypoxic conditions. In another example, U.S. Pat. No. 5,801,054 describes a growth vessel with a self-contained atmosphere. The vessel described therein allows culturing of cells under desired gas concentrations.

Once confluent, additional proliferative signals lead to cell death in the culture. Therefore, it is preferred that, once the cells are confluent or near confluent, the medium is changed to a medium that lacks proliferative agents. This may be accomplished by using the growth medium without the addition of serum. Although lacking serum, the medium may contain components that maintain the survival of the IMR-32 cells. Such components are commercially available as B27 (Gibco).

After the medium change, the cells are incubated (e.g., 37° C. in 5% CO₂ with the balance being atmospheric air) to allow expression and production of APP and the products of proteolytic processing of the APP protein. Whereas previous methods describe a 48 hr incubation period (Asami-Odaka, 1995), the methods of the present invention allow for much shorter incubation periods. Although incubations of 48 hrs or longer can be used, the high yield of peptides resulting from the proteolytic processing of APP obtained by the methods of the present invention allow for detectable levels of peptides in less than 4 hrs depending on the detection method used.

The present invention includes a method of making an IMR-32 cell culture for the production of APP and the peptides resulting from the proteolytic processing of APP. Such cell culture has novel characteristics in that the cells of the culture collectively create a yield of peptides resulting from the proteolytic processing of APP (per well or ml of culture medium) higher than the yield obtained from IMR-32 cell cultures created by low density plating. For example, the yield of Aβ₁₋₄₀ and Aβ₁₋₄₂ from IMR-32 cell cultures created in one embodiment of high density plating at 24 hrs is about 2200 pg/ml (1375 pg/cm² of growth area) and about 300 pg/ml (187.5 pg/cm² of growth area), respectively. In another embodiment, the yield of Aβ₁₋₄₀ and Aβ₁₋₄₂ from IMR-32 cell cultures of high density plating at 24 hrs is about 1100 pg/ml (687.5 pg/cm² of growth area) and about 150 pg/ml (93.75 pg/cm² of growth area), respectively

In certain aspects of the invention, one or more peptides resulting from the proteolytic processing of APP are collected. Because certain peptides resulting from the proteolytic processing of APP are secreted by the cells into the surrounding medium, collection may be as simple as removing the medium from the cells. In other embodiments, the cells are collected and lysed to obtain intracellular APP and/or peptides resulting from the proteolytic processing of APP. After collection, additional steps may be taken to quantify, purify, or dilute one or more products.

Methods of purifying peptides are well known in the art. For example, a sample containing one or more peptides resulting from the proteolytic processing of APP may be run over an affinity-column containing a component that binds to one or more products. Depending on what is desired, the binding component may be specific to a particular product or may be cross-reactive with more than one product. After binding, the one or more products are eluted from the affinity column and collected.

In certain embodiments, the presence of a product within the collected sample may be detected. Methods of detecting peptides within a sample are well known in the art and include immunoassays, NMR, gel electrophoresis, and Western blotting.

In an exemplary embodiment, ELISA is used to detect the presence of one or more products within a sample. Antibodies to detect APP and peptides resulting from the proteolytic processing of APP are well known and commercially available (Examples include Calbiochem Cat. No. PC149, PC150, and PC 152 (Aβ₁₋₄₀, Aβ₁₋₄₂, and Aβ₁₋₄₃, respectively; Cambio Cat No. CA-4786.600 (APP and sAPP-α). Additional examples are provided in Table A.

TABLE A

| Antibody | Manufacturer | Target |
|---|---|---|
| LN27 | Zymed Labs (Cat. # 13-0200) | APP |
| 22C11 | Boehringer Mannheim (Cat. # 1285262) | APP |
| 3E9 | Calbiochem (Cat. # 171537) | APP |
| 6E10 | Signet Labs (Cat. # 9320, #9340) | Aβ₁₋₄₀ |
| | | Aβ₁₋₄₂ |
| 4G8 | Signet Labs (Cat. # 9220, # 9240) | Aβ₁₋₄₀ |
| | | Aβ₁₋₄₂ |
| R162 | NY Institute Basic Research (Cat. # R162) | Aβ₁₋₄₀ |
| R165 | NY Institute Basic Research (Cat. # R165) | Aβ₁₋₄₂ |
| R226 | NY Institute Basic Research (Cat. # R226) | Aβ₁₋₄₂ |

The methods of the present invention are particularly useful in methods of producing peptides resulting from the proteolytic processing of APP. Aβ peptides are potent neurotoxins due to their ability to inhibit sodium channel activity as described in co-owned International Patent Publication WO 01/46700. Further cytotoxicity assays utilizing Aβ peptides are known in the art (Hartley et al., J. Neuroscience 19:8876-84, 1999; Pike, J. Neuroscience 13:1676-87, 1993). Because large quantities of peptide are needed to perform Aβ cytotoxicity assays, the methods of the present invention are particularly useful for producing such large quantities of Aβ peptides.

The methods of the present invention greatly decrease the amount of incubation time needed to obtain detectable levels of peptides resulting from the proteolytic processing of APP in the IMR-32 cells or the surrounding medium. This reduced incubation time allows for faster turnaround time when screening for modulators of APP processing. Generally, screening for modulators involves incubating the IMR-32 cells in medium containing a candidate modulator, followed by detecting one or more peptides resulting from the proteolytic processing of APP secreted by the cells into the medium or within a cell lysate to create a measurement. Although the measurement can be used to produce an approximate concentration of one or more peptides in the sample, it also may be used to compare relatively to another sample, for example, a control measurement. The control measurement may be that obtained using a known inhibitor of the production of one or more products. Alternatively, the control measurement may be obtained by incubating the IMR-32 cells in medium that lacks the candidate modulator or any other inhibitor. In certain embodiments, measurements are made at several concentrations of the candidate compound, and dose-dependent effects of the candidate compound on APP processing are measured.

Depending on the concentration effect or comparison to control measurements, a candidate compound may be found to be an inhibitor or an inducer of the production of one or more peptides resulting from the proteolytic processing of APP (FIG. 1). For example, after high-density seeding and growth to confluency or near confluency of IMR-32 cells in several wells of a tissue culture plate, the media may be removed and serum-free media containing a range of concentrations (including a concentration of 0) of the candidate modulator of Aβ production may be added to the cells, with separate wells containing different concentrations of the candidate modulator. One or more wells including a known inducer or inhibitor of Aβ production can be included. An inhibitor is characterized by causing a dose-dependent decrease of Aβ production by the IMR-32 cells, whereas an inducer is characterized by causing a dose-dependent increase in Aβ production.

Of course, additional measurements or characterizations of the IMR-32 cells can be made to better understand or characterize the modulator. For example, sAPP-α production could be measured. sAPP-α results from the cleavage of APP by α-secretase and is not believed to be associated with the formation of amyloid plaques. Moreover, the cleavage site of α-secretase is within the region of APP that forms the Aβ peptides. Thus, an inhibitor of sAPP-α production (e.g., an inhibitor of α-secretase) may have the effect of inducing Aβ production, whereas, conversely, an inducer of sAPP-α production may have the effect of inhibiting Aβ production.

The level of APP expression by the IMR-32 cells can be measured. Such measurement helps to determine whether the inducer or inhibitor of the production of one or more peptides resulting from the proteolytic processing of APP causes its effect by affecting APP expression or whether it is affecting the activity of a secretase. Those inhibitors or inducers that do not affect APP expression can be further characterized by determining their effect on one or more secretases. For example, the compound may be tested for its effect on the secretases that produce the Aβ peptides from APP as described in published U.S. patent application US-2001-0016324, published Aug. 23, 2001.

The level of cell death can be measured. In certain circumstances, the inhibitor of the production of one or more products may be causing inhibition by causing cell death. Thus, rather than being a specific inhibitor of the production of APP-associated products, the inhibitor may be reducing global protein production in the IMR-32 cells. Measurement of non-APP-associated products can be useful in determining this effect, also.

Methods of measuring cell death (or viability) are well known in the art. Examples include trypan blue exclusion, measuring LDH release, detecting DNA content, and measuring superoxide dismutase activity using WST-1, (Peskin and Winterbourn, Clin Chim Acta 293(1-2):157-66, 2000).

The methods of the present invention provide information about one or more candidate modulators. This information is useful for many reasons. For example, once a particular modulator is screened, the results of the screen may be placed into a report. By distributing the report, unnecessary repeating of the screen by another group is prevented. Furthermore, the reports of many screening events may be collected and analyzed to look for common features among modulators having similar activity (e.g., inhibitors of C peptide production). The report may be in essentially any form including electronic, such as an e-mail, spreadsheet, or word processing file; paper, such as a printout, laboratory notebook, or research summary; and oral, such as by telephone, conference, or audio recording.

A compound found to be a modulator of processing of APP can be evaluated or further characterized in other in vitro or in vivo assays. Alternatively, a compound suspected to modulate processing of APP based on the result of other in vitro or in vivo assays may be further evaluated or characterized using the methods of the present invention. Thus, the screening assays of the present invention may be used in conjunction with other screening methods in confirming or further evaluating the activity of a candidate modulator.

Additional assays are known in the art, some of which are described in U.S. Pat. Nos. 6,153,171, 6,211,235, and WO 01/23533 (incorporated herein by reference in their entirety). One such in vitro assay uses cultured human glioblastoma cell lines transfected with DNA encoding either a wild-type 695 amino acid isoform of APP or a mutated APP that contains changes that appear to make the molecule more susceptible to proteolytic cleavage that results in increased production of Aβ peptides (Mullan et al., Nature Genet. 1:345-347, 1992). In performing this assay, a modulator or candidate modulator is added to the culture medium and, after a selected period of time, the culture medium and/or cell lysates are analyzed using immunochemical assays to detect the relative amounts of Aβ peptides, total soluble APP (sAPP), a portion of sAPP designated α-sAPP, and C-terminal fragments of APP. In one particular embodiment, the culture medium and cell lysates are analyzed by immunoblotting coupled with laser scanning densitometry and ELISAs using several different antibodies. A positive test for an inhibitor of Aβ peptide production occurs when: (1) there is a decrease in the Aβ peptide in the medium relative to control cultures; and/or (2) the relative amount of total sAPP in the medium increases; and/or (3) there is a decrease in the amount of C-terminal amyloidogenic fragments larger than 9 kDa and smaller than 22 kDa in the cell lysate as a result of differential processing; and/or (4) there is an increase in the amount of α-sAPP in the medium relative to control cultures. Control cultures can be cultures that have not been contacted with the compound. The Aβ assay can be done using cells (e.g., HGB 717/Swed) that have been transfected with DNA encoding the mutated APP. Alternatively, the assays are performed using cells, such as HGB695 cells, that have been transfected with DNA encoding a wild-type APP.

In other in vitro assays, isolated, recombinant secretases are incubated with substrate in the presence of a candidate modulator and the effect of the candidate modulator on the ability of the secretase to cleave the substrate is determined. Examples of such assays are disclosed in WO 01/23533.

The ability of compounds to modulate processing of APP can also be evaluated in vivo using an animal model for Alzheimer's disease. Such animal models, and their use in screening assays, are well known in the art and include non-transgenic animal models (Kowall et at. Proc. Natl. Acad. Sci. U.S.A. 88:7247-7251, 1991; U.S. Pat. No. 6,172,277 (incorporated herein by reference in their entirety)) and transgenic animal models (U.S. Pat. Nos. 6,245,964, 6,211,428, 6,211,235, 6,184,435, 6,175,057, 6,037,521,5,912,410, 5,877,399, and 5,849,999(incorporated herein by reference in their entirety)). Compounds can be administered through a number of methods including through a canula implanted in the cranium of the test animal (Lamb et al. Nature Genet. 5:22-29, 1993; Pearson et al. Proc. Natl. Acad. Sci. U.S.A. 90:10578-10582, 1993). After a predetermined period of administration, the animals are tested for amyloid formation or lack thereof. Often the animals are sacrificed. The hippocampi are evaluated in immunoblot assays or other suitable assays to determine the relative level of APP-associated products compared to untreated control animals.

Polypeptides and cells produced by a method of the present invention are also useful in neurotoxicity assays Many different neurotoxicity assays are known in the art (Wang et al., J. Biol Chem 276(45):42027-34, 2001; Liu and Piasecki, Anal Biochem 289(2):130-6, 2001; Tang and Zhang, Acta Pharmacol Sin 22(4):380-384, 2001; Heo et al., Amyloid 8(3):194-2001, 2001).

Modulators may be used to create pharmaceutical compositions. The compound itself may be formulated into a pharmaceutical composition or knowledge of the structure of the modulator can be used by pharmaceutical chemists to design a pharmaceutical. Such compositions are useful for further in vivo analysis of the modulator's properties and also for therapy in a suitable animal model. Compositions are provided that contain therapeutically effective amounts of a modulator. The compounds are preferably formulated into suitable pharmaceutical preparations such as tablets, capsules or elixirs, for oral administration or in sterile solutions or suspensions for parenteral administration, as well as transdermal patch preparation. Typically, the modulators identified by the screening method of the present invention are formulated into pharmaceutical compositions using techniques and procedures well known in the art.

For example, about 10 to 500 mg of a modulator or a physiologically acceptable salt thereof is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a desired effect is obtained.

To prepare compositions, one or more modulator is mixed with a suitable pharmaceutically acceptable carrier. Upon mixing or addition of the compound(s), the resulting mixture may be a solution, suspension, emulsion or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action or have other action. The compounds may be formulated as the sole pharmaceutically active ingredient in the composition or may be combined with other active ingredients.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as tween, or dissolution in aqueous sodium bicarbonate. Derivatives of the compounds, such as salts of the compounds or prodrugs of the compounds may also be used in formulating effective pharmaceutical compositions.

The concentrations of the compounds are effective for delivery of an amount, upon administration, that ameliorates the symptoms of the disorder for which the compounds are administered. Typically, the compositions are formulated for single dosage administration.

Preferably, the active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compositions can be enclosed in ampules, disposable syringes or multiple or single dose vials made of glass, plastic or other suitable material. Such enclosed compositions can be provided in kits.

The concentration of active compound in the drug composition will depend on absorption, inactivation and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules or troches. Pharmaceutically compatible binding agents and adjuvant materials can be included as part of the composition.

The tablets, pills, capsules, troches and the like can contain any of the following ingredients, or compounds of a similar nature: a binder, such as, but not limited to, gum tragacanth, acacia, corn starch or gelatin; an excipient such as microcrystalline cellulose, starch and lactose, a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, and fruit flavoring.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings and flavors.

Solutions or suspensions used for parenteral, intradermal, subcutaneous, or topical application can include any of the following components: a sterile diluent, such as water for injection, saline solution, fixed oil, a naturally occurring vegetable oil like sesame oil, coconut oil, peanut oil, cottonseed oil, etc. or a synthetic fatty vehicle like ethyl oleate or the like, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or multiple dose vials made of glass, plastic or other suitable material. Buffers, preservatives, antioxidants and the like can be incorporated as required.

If administered intravenously, suitable carriers include physiological saline or phosphate buffered saline (PBS), and solutions containing thickening and solubilizing agents, such as glucose, polyethylene glycol, and polypropylene glycol and mixtures thereof. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811.

The active compounds may be prepared With carriers that protect the compound against rapid elimination from the body, such as time release formulations or coatings. Such carriers include controlled release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of such formulations are known to those skilled in the art.

The compounds may be formulated for local or topical application, such as for topical application to the skin and mucous membranes, such as in the eye, in the form of gels, creams, and lotions and for application to the eye or for intracisternal or intraspinal application. Such solutions, may be formulated as 0.01%-100% (weight to volume) isotonic solutions, pH about 5-7, with appropriate salts. The compounds may be formulated as aeorsols for topical application, such as by inhalation (U.S. Pat. Nos. 4,044,126, 4,414,209, and 4,364,923).

Finally, the compounds may be packaged as articles of manufacture containing packaging material, an acceptable composition containing a modulator provided herein, which is effective for treating the particular disorder, and a label that indicates that the compound or salt thereof is used for treating the disorder.

Inhibitors of Aβ peptides have pharmacological utility and also utility as reagents. It is recognized in this art that compounds that exhibit activities in assays that assess the ability of the compounds to alter or modulate the activity of proteins associated with the deposition of cerebral amyloid, are pharmacologically useful and potentially therapeutically useful in the treatment of disorders that involve such deposition.

The dose ranges, which can be established empirically, for use in the treatment of disease states will depend upon the etiology, nature, and severity of the disease state as well as such other factors as determined by the attending physician. The broad range for effective treatment is about 0.01 to 10 mg per kilogram (kg) of body weight per day. The preferred range is about 0.1 to 10 mg/kg of body weight per day.

The active compounds can be administered by any appropriate route, for example, orally, parenterally, intravenously, intradermally, subcutaneously, or topically, in liquid, semi-liquid or solid form and are formulated in a manner suitable for each route of administration. Preferred modes of administration include oral and parenteral modes of administration.

Since, it is feasible to indirectly measure the presence of, and over the course of time, to determine the rate of increase of those protein segments believed to be critical to the formation of amyloid plaques located in the brain (U.S. Pat. No. 5,270,165), dosages can be empirically determined by the physician. As these techniques involve the use of cerebrospinal fluids, such techniques, and other equivalently functioning procedures, will be useful to the attending physician in determining the need to modify the dosage for individual patients.

In treating neurodegenerative disease states, it is sufficient to start treating the patient as soon as the attending physician makes his or her diagnosis that the patient is suffering from such a disease. Thus, although the progress of treatment of the patient may be monitored by the measurements of those biological factors that characterize the diseases, it is not necessary to so-evaluate such characteristics before treatment. Rather it is within the province of the attending physician to determine when it is in the best interest of the patient to start treatment. Therefore, patients showing increased probabilities of the disease state, (e.g. by carrying known familial genetic markers that increase the probability of the incidence of neurodegenerative diseases as well as the patient's general behavioral characteristics and other indicia of these diseases) can be treated by the methods and with the compositions provided herein.

Amyloid plaques are believed to accompany and/or be involved in the process responsible for the development and progression of certain neurodegenerative disease states. Without any intent to limit—or restrict—the modulators and methods provided herein to any specific mechanism of action for the end-use applications, it is believed that the modulators effectuate a modulation of the processing of the amyloid precursor protein (APP), the progenitor of the deposited amyloidogenic Aβ peptides found in senile plaques in the brains of patients diagnosed with, for example, Alzheimer's disease. Thus, certain modulators provided herein are useful in the treatment of such neurodegenerative disease states in which such amyloid plaques accumulate or are implicated in the etiology thereof, including, but not limited to: Alzheimer's disease, cognition deficits, Down's Syndrome, Parkinson's disease, cerebral hemorrhage with amyloidosis, dementia pugilistica, head trauma and in the treatment of conditions characterized by a degradation of the neuronal cytoskeleton resulting from a thrombolytic or hemorrhagic stroke.

For example, it is believed that the modulator can be used in the treatment of Alzheimer's patients through the modulation of APP processing to effectuate a beneficial result by: (a) decreasing the formation of Aβ; (b) modulating the generation of a mutually exclusive, alternative-processed form of APP that precludes Aβ formation (α-sAPP); and/or, (c) modulating the generation of partially processed C-terminal Aβ-containing amyloidogenic peptides.

### EXAMPLES

Having generally described the invention, the same will be more readily understood by reference to the following examples, which are provided by way of illustration and are not intended as limiting.

### Example 1

This Example demonstrates the effect of plating density on Aβ peptide production by IMR-32 cells. Three different protocols were used to culture the cells and the amount of Aβ₁₋₄₀ and Aβ₁₋₄₂ peptide produced was determined at various time points after confluency.

Protocol A—9.4×10⁴ cells/cm² Density Plating—Growth in 10% Fetal Bovine Serum

On day 1, IMR-32 cells were plated at 1.5×10⁵ cells/ml, 200 μl/well (3×10⁴ cells/well or about 9.4×10⁴ cells/cm²), in a 96-well plate grown in media containing 10% fetal bovine serum (minimum essential media with Earle's salts without L-glutamine, 10% fetal bovine serum, 100 U/ml penicillin-100 μg/ml streptomycin, 1 mM sodium pyruvate, and 0.1 mM non-essential amino acids) placed in a tissue culture incubator at 37° C. in an atmosphere of 95% air 5% CO₂. On day 5, cells were confluent and fed with serum-free media supplemented with B27 (Dulbecco's modified Eagle medium, 1×B27 (Gibco), 2 mM L-glutamine, 25 mM HEPES, 100 U/ml penicillin-100 μg/ml streptomycin). Media was removed from cells at 4.75 hours, 8 hours, and 24 hours after media change. All samples were analyzed for Aβ₁₋₄₀. and run neat in a direct sandwich ELISA.

Protocol B—1.6×10⁵ cells/cm² Density Plating—Growth in 10% Fetal Bovine Serum

On day 1, cells were plated at 2.5×10⁵ cells/ml, 200 μl/well (5×10⁴ cells/well or about 1.6×10⁵ cells/cm²), in a 96-well plate grown in identical media and conditions described in Protocol A. On day 5, cells were confluent and media was replaced with serum-free media supplemented with B27 (media components described in protocol A). Media was removed at 24 hours after media change. All samples were analyzed and run neat for Aβ₁₋₄₀ and Aβ₁₋₄₂ in a direct sandwich ELISA.

Protocol C—High Cell Density Plating—Growth in 10% Heat Inactivated Fetal Bovine Serum

All procedures were performed as described in Protocol B, however the 5×10⁴ cells/well were grown in media containing 10% heat inactivated fetal bovine serum (minimim essential media with Earle's salts without L-glutamine, 10% heat inactivated fetal bovine serum, 2 mM L-glutamine, 100 U/ml penicillin-100 μg/ml streptomycin, 1 mM sodium pyruvate, 0.1 mM non-essential amino acids). Media was removed 4 hours and 24 hours after serum-free B27 media change. All samples were analyzed and run neat or diluted 1:2 in 1% BSA in PBS for Aβ₁₋₄₀ and Aβ1-42 in a direct sandwich ELISA.

Results

The results of the different protocols on Aβ peptide production are shown in Table I (Aβ₁₋₄₀) and Table II (Aβ₁₋₄₂). Protocol B, which utilizes growth medium with 10% serum that is not heat inactivated, provided the highest levels of Aβ peptides at 24 hours after media change (2182 pg/ml Aβ₁₋₄₀ and 329 pg/ml Aβ₁₋₄₂). At 24 hours, levels of Aβ peptides were 2 fold higher than levels reported by Asami-Odaka et al. (Biochemistry 34:10272-10278, 1995) for both peptides. This increase is even more striking when considering the fact that Asami-Odaka isolated media 48 hours after the media change. Furthermore, the ratio of Aβ₁₋₄₂ to total Aβ peptide (˜10%) was consistent with those previously observed with IMR-32 cells and with the ratio of the peptides in cerebrospinal fluid or plasma of normal adults (Asami-Odaka et al., 1995). Thus, higher seeding densities resulted in increased peptide yield allowing for statistically significant levels of peptide to be detected at earlier time periods than previous techniques.

TABLE I
Secreted Aβ₁₋₄₀ Levels from IMR-32 Cells

| | Sample | | | | |
|---|---|---|---|---|---|
| | Preparation | Aβ₁₋₄₀ (pg/ml) ± SE | | | |
| Protocol | for ELISA | 4 hours | 4.75 hours | 8 hours | 24 hours |
| A | Neat | np | 75 ± 2 | 134 ± 3* | 539 ± 13+ |
| B | Neat | np | np | np | 2182 ± 46 |
| C | Neat | 121 ± 2 | np | np | 1066 ± 32* |
| C | 1:2 | 161 ± 6 | np | np | 1338 ± 33* |

*p < 0.05 versus 4 hour or 4.75 hour group; + p < 0.05 versus 8 hour group; np = not performed

TABLE II

| | Sample | | | | |
|---|---|---|---|---|---|
| | Preparation | A ± ₁₋₄₂ (pg/ml)∀SE | | | |
| Protocol | for ELISA | 4 hours | 4.75 hours | 8 hours | 24 hours |
| A | Neat | np | np | np | np |
| B | Neat | np | np | np | 329 ± 14 |
| C | Neat | 10± 1 | np | np | 176 ± 11* |
| C | 1:2 | 22 ± 4 | np | np | 219 ± 8* |

*p < 0.05 versus 4;
np = not performed

### Example 2

This Example demonstrates Aβ peptide yields from high density plating increase beyond the 24 hour observation time point.

Materials and Methods

On day 1, IMR-32 cells were plated at 5×10⁴ cells/well or 1×10⁵ cells/well in a 96-well plate grown in media containing 10% fetal bovine serum or 10% heat inactivated fetal bovine serum (minimum essential media with Earle's salts w/o L-glutamine, 2 mM L-glutamine, 100 U/ml penicillin-100 μg/ml streptomycin, 1 mM sodium pyruvate, and 0.1 mM non-essential amino acids.) On day 6, cells were confluent and media was replaced with serum-free media supplemented with B27. Media was removed 4 hours, 8 hours, 24 hours, and 48 hours after serum-free B27 media change. Samples were analyzed and run neat or diluted 1:2 in 1% BSA in PBS for Aβ₁₋₄₀ or Aβ₁₋₄₂ in a direct sandwich ELISA. Intracellular soluble Aβ was determined by lysing cells in TENND buffer and run neat in a direct sandwich ELISA.

Results

As can be seen in Table III and IV, a time-dependent elevation of secreted Aβ peptides was seen from 4 hours to 48 hours. A leveling off of Aβ production was achieved at 48 hours. However, the level of Aβ peptide produced at 48 hours using the high density plating is 2 fold more than the Aβ levels documented in the literature (Asami-Odaka, 1995) at 48 hours. There was no difference in Aβ peptide levels when comparing 5×10⁴ and 1×10⁵ plating at 4, 8, 24, and 48 hours (Tables III and IV). Additionally, cells grown in normal and heat inactivated serum provided similar levels of secreted Aβ peptide (Tables III and IV).

TABLE III
Secreted Aβ₁₋₄₀ Levels

| | | Aβ₁₋₄₀ (pg/ml) | | | | | |
|---|---|---|---|---|---|---|---|
| | | | | | 24 hours | | 48 hours |
| IMR-32 | Growth Serum | 4 hours | 8 hours | 24 hours | (1:2) | 48 hours | (1:2) |
| 5 × 10⁴ cells/well | Normal | 173 | 365 | 2140 | 1912 | 2871 | 3807 |
| 1 × 10⁵ cells/well | | 147 | 352 | 1950 | 2013 | 2491 | 2452 |
| 5 × 10⁴ cells/well | Heat Inactivated | 223 | 386 | 2551 | 3318 | 2778 | 2547 |
| 1 × 10⁵ cells/well | | 128 | 355 | 1577 | 1721 | 2556 | 2517 |

| | | | | |
|---|---|---|---|---|
| Mean | 168 ± 21 | 365 ± 8 | 2148 ± 196 | 2752 ± 159 |

TABLE IV
Secreted Aμ₁₋₄₂ Levels

| | | Aβ₁₋₄₂ (pg/ml) | | | | | |
|---|---|---|---|---|---|---|---|
| | | | | | 24 hours | | 48 hours |
| IMR-32 | Growth Serum | 4 hours | 8 hours | 24 hours | (1:2) | 48 hours | (1:2) |
| 5 × 10⁴ cells/well | Normal | 18 | 41 | 259 | 246 | 284 | 422 |
| 1 × 10⁵ cells/well | | 14 | 38 | 220 | 241 | 244 | 304 |
| 5 × 10⁴ cells/well | Heat Inactivated | 22 | 42 | 251 | 277 | 283 | 407 |
| 1 × 10⁵ cells/well | | 12 | 37 | 177 | 233 | 250 | 335 |

| | | | | |
|---|---|---|---|---|
| Mean | 17 ± 2 | 40 ± 1 | 238 ± 11 | 316 ± 24 |

IMR-32 cells maintained stable intracellular Aβ peptide levels which were 3 to 41 fold lower than secreted Aβ1-40 levels, and 2 to 32 fold lower than secreted Aβ₁₋₄₂ levels (Tables V and VI). Thus, in methods using IMR-32 cells to characterize or identify a BACE inhibitor, the inhibitor would most accurately be characterized or identified by quantifying Aβ peptide levels secreted from the cells.

TABLE V
Intracellular Soluble Aμ₁₋₄₀ Levels

| | | Aμ₁₋₄₀ Levels (pg/ml) | | | |
|---|---|---|---|---|---|
| IMR-32 | Growth Serum | 4 hours | 8 hours | 24 hours | 48 hours |
| 5 × 10⁴ | Normal | 69 | 76 | 59 | 51 |
| cells/well | | | | | |
| 1 × 10⁵ | | 75 | 81 | 65 | 60 |
| cells/well | | | | | |
| 5 × 10⁴ | Heat Inactivated | 68 | na | 70 | 58 |
| cells/well | | | | | |
| 1 × 10⁵ | | 70 | 91 | 60 | 69 |
| cells/well | | | | | |
| Mean | | 71 ± 2 | 77 ± 7 | 63 ± 2 | 59 ± 4 |

TABLE VI
Intracellular Soluble Aμ₁₋₄₂ Levels

| | | Aμ₁₋₄₂ Levels (pg/ml) | | | |
|---|---|---|---|---|---|
| IMR-32 | Growth Serum | 4 hours | 8 hours | 24 hours | 48 hours |
| 5 × 10⁴ | Normal | 11 | 6 | 14 | 9 |
| cells/well | | | | | |
| 1 × 10⁵ | | 8 | 10 | 11 | 11 |
| cells/well | | | | | |
| 5 × 10⁴ | Heat Inactivated | 10 | 15 | 9 | 9 |
| cells/well | | | | | |
| 1 ×10⁵ | | 10 | 14 | 8 | 14 |
| cells/well | | | | | |
| Mean | | 10 ± 1 | 10 ± 3 | 11 ± 1 | 11 ± 1 |

### Example 3

This Example describes how to isolate and detect soluble APP using IMR-32 cells seeded at a high density.

Cell Plating for Intracellular Aβ Determinations

On day 1, cells are plated at 5×10⁵ cells/well or 1×10⁶ cells/well in a 6-well culture plate (9.4 cm²/well Coming Costar 6-well polystyrene) in growth media containing fetal bovine serum that was or was not heat inactivated (MEM, 10% serum, 2 mM L-glutamine, 100 U/ml-100 μg/ml penicillin/streptomycin, 1 mM NaPyruvate, 0.1 mM non-essential amino acids). On day 5, cells are fed with growth media. On day 6, media is replaced with serum-free media supplemented with B27 (DMEM, 1×B27, 2 mM L-glutamine, 25 mM HEPES, 100 U/ml-100 μg/ml penicillin/streptomycin). Intracellular Aβ is obtained 4, 8, 24 and 48 hours after media change.

Isolation of Intracellular Soluble Aβ

Media is removed from 3 wells from the 6-well plate. Cells are rinsed 2× with 1 ml cold 1XDPBS w/o Ca²⁺, w/o Mg²⁺. Cell lysis buffer (10 mM Tris-HCl (pH 7.5), 5 mM EDTA, 150 mM NaCl, 1% v/v NP-40, and 0.5 % w/v deoxycholate) is added to each of the 3-wells (100 μl/well). Cells are scraped with a cell scraper and pooled from 3 wells. The mixture is transferred to a microfuge tube and spun at 134000 rpm at 4° C. for 10 min. The resulting supernatants are stored at 20° C. until needed for use.

Detection of Secreted Aβ in Conditioned Media and Intracellular Soluble Aβ by a Direct Sandwich ELISA

Synthetic purified Aβ₁₋₄₀ and Aβ₁₋₄₂ (BACHEM) and Aβ in samples are captured by coating 96-well ½ area flat bottom high binding ELISA plates (Corning Costar) with mouse monoclonal antibody 6E10 (4 μg/ml, Senetek) prepared in 0.1 M NaHCO, pH 9.6 at 4° C. incubated overnight. Plates are blocked overnight at 4EC with 1% bovine serum albumin (BSA, Sigma) in Dulbecco's PBS (Pierce) with 0.05% Tween-20 (DPBST). Aβ peptides are diluted in B27 serum-free media for the detection of secreted Aβ, and cell lysis buffer for the detection of soluble Aβ peptides. Antigens in conditioned media plates are incubated overnight at 4° C.; intracellular soluble antigens are incubated for 3 hours at room temperature. Peptides are detected with biotinylated rabbit polyclonal antibodies R162 (1/2400) for Aβ₁₋₄₀ and R165 (1/1000) for Aβ₁₋₄₂, both obtained from Pankaj Mehta from the Institute for Basic Research, Staten Island, N.Y.; incubated overnight at 4° C. Neutravidin conjugated with horseradish peroxidase (0.1 μg/ml, Pierce) is reacted with biotin for 30 min. Addition of TMB substrate (Kirkegaard & Perry) for ˜30 minutes produces a colored-end product when converted by HRP, and the reaction is stopped using 1 M H₃PO₄. The colored end-point is detected at an absorbance of 450 nm using a 96-well plate reader.

Alpha-Secretase Cleavage of Amyloid Precursor Protein (APP) Produces a Soluble Fragment of APP (sAPP-α) Detectable in Conditioned Media from IMR-32 Cells

APP is cleaved by α-secretase within the Aβ region, therefore precluding production of amyloidogenic products. In addition, cleavage of APP by α-secretase releases a large soluble non-amyloidogenic peptide, termed sAPP-α, which has neurotrophic properties (Roch et al., Proc. Natl. Acad. Sci. USA 91:7450-4, 1994) and has been show to improve cognitive functions in rodents and humans (Almkvist et al., Arch. Neurol. 54:641-4, 1997, Roch et al., 1994). The IMR-32 cell line has detectable quantities of sAPP-α (0.1 μg/ml) determinable by a direct sandwich ELISA. Media is removed after cells are confluent, and replaced with serum containing media. Conditioned media is analyzed 48 hours after media change.

The direct sandwich ELISA protocol is performed as follows: ELISA 96-well plates (Corning Costar) are coated with mouse monoclonal antibody LN27 (0.1 μg/ml, Zymed Laboratories) prepared in 0.1 M NaHCO₃, pH 9.6 at 4° C. incubated overnight. Plates are subsequently blocked overnight at 4° C. with 1% BSA in DPBST. Synthetic sAPP-α (prepared at Pharmacia Corp., USA) is diluted in IMR-32 growth media, and all antigens are incubated overnight at 4° C. Biotin labeled mouse monoclonal antibody 6E10 (1 μg/ml, Senetek), prepared in 1%. BSA in DPBSST, is added and incubated overnight at 4° C. Neutravidin conjugated to horseradish peroxidase (HRP; 0.1 μg/ml, Pierce) is reacted with biotin for 30 minutes. Addition of TMB substrate (Kirkegaard & Perry) for 50 to 60 minutes produces a colored end product when converted by HRP, and the reaction is stopped using 1 M H₃PO₄. The colored end point is detected at an absorbance of 450 nm using a 96-well plate reader.

While the present invention has been described in terms of specific embodiments, it is understood that variations and modifications will occur to those in the art, all of which are intended as aspects of the present invention. Accordingly, only such limitations as appear in the claims should be placed on the invention.

1. A method of culturing IMR-32 cells to increase production of peptides resulting from the proteolytic processing of amyloid precursor protein in the cells, the method comprising steps of:
(a) seeding a culture vessel with EMR-32 neuroblastoma cells at a density of at least about 5.0×10⁴ cells/cm² of growth area to about 1.0×10⁶ cells/cm² of growth area;
(b) growing the IMR-32 neuroblastoma cells to confluency or near confluency in medium containing serum;
(c) changing the medium to serum-free medium; and
(d) incubating the IMR-32 neuroblastoma cells in the serum-free medium, wherein, during incubation in the serum-free medium, the IMR-32 cells produce peptides resulting from the proteolytic processing of amyloid precursor protein.

2. The method of claim 1, wherein in step (a) the IMR-32 neuroblastoma cells are seeded at a density of about 2.5×10⁵ to about 5×10⁵ cells/ml.

3. The method of claim 1, wherein in step (b) the IMR-32 neuroblastoma cells are grown until the cells occupy at least 80% of the growth area.

4. The method of claim 3, wherein in step (b) the IMR-32 neuroblastoma cells are grown until the cells occupy approximately 100% of the growth area.

5. The method of claim 1, wherein the IMR-32 neuroblastoma cells are incubated in the serum-free medium for less than 24 hours.

6. The method of claim 5, wherein the IMR-32 neuroblastoma cells are incubated in the serum-free medium for between 4 and 24 hours.

7. The method of claim 1, wherein in step (d) the cells are incubated for a time sufficient to create a concentration of the amyloid beta peptides in the medium of at least 3000 pg/ml.

8. The method of claim 1, further comprising a step of collecting the serum free medium after the incubation time.

9. The method of claim 8, further comprising the step of measuring the amount of amyloid beta peptides in the collected serum-free medium.

10. The method of claim 1, wherein the serum-free medium is B27 supplemented medium.

11. The method of claim 1, wherein the serum of step (b) is heat inactivated serum.

12. A method of screening for a modulator of proteolytic processing of amyloid precursor protein, the method comprising the steps of:
(a) seeding a culture vessel with IMR-32 neuroblastoma cells at a density of at least about 5.0×10⁴ cells/cm² of growth area to about 1.0×10⁶ cells/cm² of growth area;
(b) growing the IMR-32 neuroblastoma cells in medium containing serum;
(c) changing the medium to serum-free medium;
(d) incubating the IMR-32 neuroblastoma cells in the serum-free medium, wherein the serum-free medium comprises a candidate modulator of proteolytic processing of amyloid precursor protein;
(e) measuring at least one peptide in the serum-free medium produced by proteolytic processing of amyloid precursor protein by the cells; and
(f) comparing the measurement of step (e) to a control measurement.

13. The method of claim 12, wherein a lower measurement of step (e) than the control measurement for a peptide produced by proteolytic processing of amyloid precursor protein indicates an inhibitor of the production of that peptide.

14. The method of claim 12, wherein the measuring comprises a determination of amyloid beta peptide concentration in the medium.

15. The method of claim 14, wherein the concentration of Aβ₁₋₄₀ peptide is determined.

16. The method of claim 14, wherein the concentration of Aβ₁₋₄₂ peptide is determined.

17. The method of claim 12, wherein the measuring comprises a determination of soluble amyloid precursor protein alpha concentration in the medium.

18. The method of claim 12, wherein incubation is less than 4 hours.

19. The method of claim 12, wherein the control measurement comprises a measurement of at least one peptide produced by proteolytic processing of amyloid precursor protein in the absence of the candidate modulator in the incubating step.

20. A method of detecting an inhibitor of amyloid beta peptide production, the method comprising the steps of:
(a) seeding a culture vessel with IMR-32 neuroblastoma cells at a density of at least about 5.0×10⁴ cells/cm² of growth area to about 1.0×10⁶ cells/cm² of growth area;
(b) growing the IMR-32 neuroblastoma cells in medium containing serum;
(c) changing the medium to serum-free medium;
(d) incubating the IMR-32 neuroblastoma cells in the serum-free medium, wherein the serum-free medium comprises a first concentration of a candidate inhibitor of amyloid beta peptide production;
(e) measuring a first level of amyloid beta peptide in the serum-free medium containing the concentration of the candidate inhibitor;
(f) repeating steps (a)-(e) one or more times wherein the concentration of the candidate inhibitor is different than that of the first concentration, wherein a level of amyloid beta peptide is measured for each concentration; and
(g) comparing the level of amyloid beta peptide at two or more concentrations of the candidate inhibitor, wherein a lower level amyloid beta peptide at a higher concentration of the candidate inhibitor indicates an inhibitor of amyloid beta peptide production.

21. The method of claim 20, further comprising the step of producing a composition containing an effective amount of an inhibitor detected by steps (a)-(g) in a pharmaceutical carrier.

22. The method of claim 21, further comprising the steps of administering the composition to an animal and evaluating the animal to determine amyloid beta peptide production.

23. The method of claim 20, wherein steps (a)-(e) and step (f) are performed in parallel.

24. A method of indicating an inhibitor of amyloid beta peptide production, the method comprising:
(a) detecting an inhibitor by the method of claim 14; and
(b) producing a report indicating the inhibitor.

25. A cell line produced by:
(a) seeding a culture vessel with IMR-32 neuroblastoma cells at a density of at least about 5.0×10⁴ cells/cm² of growth area to about 1.0×10⁶ cells/cm² of growth area;
(b) growing the IMR-32 neuroblastoma cells in medium containing serum until confluent or near confluent;
(c) changing the medium to serum-free medium;
(d) incubating the IMR-32 neuroblastoma cells in the serum-free medium, wherein the IMR-32 neuroblastoma cells secrete amyloid beta peptides into the medium at a rate of at least 1300 pg/ml/day.
